(19) United States
(12) Patent Application Publication
(10) Pub. No.: US 2005/0031538 A1
(43) Pub. Date: Feb. 10, 2005

(54) Neural cell assay

(76) Inventors: Dennis A. Steindler, Gainesville, FL (US); Eric D. Laywell, Gainesville, FL (US); Tong Zheng, Gainesville, FL (US)

Correspondence Address: EDWARDS & ANGELL, LLP, P.O. BOX 55874, BOSTON, MA, 02205, US

(21) Appl. No.: 10/912,472

(22) Filed: Aug. 5, 2004

Related U.S. Application Data
(60) Provisional application No. 60/492,506, filed on Aug. 5, 2003.

(51) Int. Cl. G01N 33/53; G01N 33/567; A61K 49/00

(52) U.S. Cl. 424/9.2; 435/7.2

(57) ABSTRACT

Methods and assay systems for analyzing effects of chemical and cellular agents on brain cell neurogenesis in vivo, comprising administering an agent to a test animal and determining responses of cells of brain marrow tissues, including irradiated brain marrow tissue depleted of neurogenic stems cells, and cells in brain marrow-derived neurospheres cultured in vitro.

### CROSS REFERENCE TO RELATED APPLICATIONS

The present application claims priority from U.S. Provisional Patent Application No. 60/492,506 entitled “Neural Cell Assay,” as filed on Aug. 5, 2003, the disclosure of which is incorporated by reference in its entirety.

### STATEMENT REGARDING FEDERALLY SPONSORED RESEARCH

The present invention was made with United States government support under grant NINDS37556 awarded by the National Institutes of Health. The United States government may have rights in the invention.

### FIELD OF THE INVENTION

The invention relates generally to the fields of medicine, neurology, cell biology and toxicology. More particularly, the invention relates to assays and methods for analyzing effects of an agent on neural cells, particularly cells involved in neurogenesis.

### BACKGROUND OF THE INVENTION

Persistent neurogenesis due to the cycling of multipotent neural stem cells is now recognized as a normal, homeostatic function within some mature mammalian neural structures, including the subependymal zone (SEZ), its continuous rostral migratory stream (RMS), and the hippocampus. The SEZ and hippocampus of the developing and adult mammalian brain are referred to as “brain marrow” and are sensitive barometers of the brain's status since their persistent generation of new neurons from an indigenous population of stem/progenitor cells is sensitive to genetic and epigenetic (including environmental) factors and cues that can affect the brain.

Exposure to pharmacological or toxicological agents or other agents such as food additives could interfere with brain marrow neuropoiesis, resulting in severe consequences for brain functioning, including impaired cognition and memory. As the incidence of neurological disorders such as Alzheimer's disease, Parkinson's disease and AIDS-related conditions increases, there is further recognition of an urgent need for new therapeutic agents that could increase the brain's capacity for neurogenesis. Therefore a need exists for a sensitive, quantitative assay of brain marrow neurogenic status following exposure of brain cells to various agents that can affect brain marrow cells.

### SUMMARY OF THE INVENTION

The invention relates to methods and assays for identifying and analyzing agents that have a modulating effect on brain cells by examining neurogenesis (neuropoiesis). One method of the invention includes the steps of: (a) administering the agent to a test animal; (b) determining at least one characteristic of cells in brain tissue from the SEZ, continuous RMS, olfactory bulb and/or the hippocampus; and (c) comparing the at least one characteristic of the cells to that characteristic in cells from the same area in a control animal that has not been administered the agent. A difference in the characteristic between the test animal and the control animal indicates that the agent affects brain cells.

The agent can include one or more substances such as a drug, a small molecule, a peptide, a nucleic acid or a nucleoside analog, or a cell such as a stem cell. The nucleoside analog can be selected from azidothymidine, dideoxyinosine, dideoxythymidine, dideoxycytidine, and cytosine arabinoside.

The agent can also include a force such as radiation.

The characteristics of brain tissue analyzed can include mitotic index, expression of cellular markers, migration of neuroblasts in the brain marrow, apoptosis and necrosis.

In a variation of the foregoing method, the brain tissue sample is dissociated and placed into tissue culture, preferably under conditions that promote neurosphere formation. The step of determining at least one characteristic of the cells is performed by analyzing numbers of neurospheres formed in culture and/or the cellular makeup of the neurospheres.

A variation is a method for analyzing the effect of an agent on neurogenesis in an animal that includes the steps of: (a) administering at least one stem cell to a test animal; (b) administering the agent to the stem cell or to the test animal; (c) analyzing population of the test animal's brain marrow with the administered stem cell; and (d) comparing the population in the test animal to that in a control animal administered with a control stem cell, wherein neither the control stem cell nor the control animal are administered the agent. A difference in the population by stem cells between the test animal and the control animal indicates that the agent has an effect on neurogenesis in the animal.

In the foregoing method, the stem cell can include a detectable label, and the step of analyzing population of the test and control animal's brain marrow can be performed by quantifying the amount of detectable label associated with a tissue sample of the test or control animal taken from at least one of the SEZ, continuous RMS, olfactory bulb or hippocampus.

The stem cell can be a neural stem cell, a somatic stem cell from a non-neuronal tissue or an embryonic stem cell. The stem cell can express a cell surface marker such as CD15, CD133 or CD44. The stem cell can be genetically modified.

Yet another variation of the invention is a method for increasing neurogenesis in an animal that includes the steps of: (a) depleting brain marrow in a test animal; (b) administering at least one stem cell to the test animal to repopulate the brain marrow; and (c) comparing the repopulation of the brain marrow in the test animal to that in a control animal receiving at least one stem cell without depletion of its brain marrow, wherein the presence of a greater number of neurogenic cells in said test animal indicates that neurogenesis is increased.

As in the previously described method, the stem cells can include a detectable label, and the step of analyzing repopulation of the test animal's brain marrow can be performed by quantifying the amount of detectable label associated with a tissue sample taken from at least one of the SEZ, continuous RMS, olfactory bulb or the hippocampus.

Some versions of the methods can be used to identify or analyze agents that can prevent or reduce the depletion of the brain marrow that can occur following an insult such as radiation. In this method the brain marrow is depleted, before or after the addition of a candidate protective agent, and agents are selected on the basis of their effect of preventing or reducing the amount of brain marrow depletion.

Unless otherwise defined, all technical terms used herein have the same meaning as commonly understood by one of ordinary skill in the art to which this invention belongs.

The term “drug,” as used herein, refers to any substance of potential medical use in human beings or other animals. Encompassed within this definition are compound analogs, naturally occurring, synthetic and recombinant pharmaceuticals, hormones, nucleic acids, polypeptides, neurotransmitters, etc.

The term “stem cell,” as used herein refers to a single cell capable of producing all cell types of a particular organ for the life of an animal via asymmetrical division, in which both an exact duplicate of the stem cell and a lineage-committed progenitor daughter cell is generated. Typically a stem cell can reconstitute its native niche following transplantation. Stem cells can be found in many tissues of the body, including embryonic tissues, and can include but are not limited to: embryonic stem cells and stem cells of the hematopoetic system, brain and nervous system, epithelia, epidermis, heart and cardiovascular systems, liver, gastrointestinal tract, and reproductive systems.

By the phrase “neural stem cell” is meant a cell capable of differentiating into mature neural cells such as neurons, astrocytes, and oligodendrocytes. A “stem or progenitor cell” can include any immature cell that has the attributes of a stem cell/progenitor cell that can give rise to different cell types of mature tissues and organs in the body. In general, a stem/progenitor cell is a precursor cell able to generate copies of itself, as well as give rise to more differentiated daughter cells. A stem/progenitor cell generally acts as a clonogenic cell that can respond to injury and disease in tissues of the body with repopulation attempts.

By “brain marrow” is meant those regions of the vertebrate brain in which persistent neurogenesis can occur, including the periventricular subependymal zone (SEZ) of the neuraxis, the telencephalic rostral migratory stream (RMS), the hippocampus, the olfactory bulb and discrete areas of the normal or injured central nervous system.

The term “neuropoiesis,” as used herein, is synonymous with “neurogenesis,” meaning the generation of neurons and/or glia, generally on a persistent basis.

Although methods and materials similar or equivalent to those described herein can be used in the practice or testing of the present invention, suitable methods and materials are described below. All publications, patent applications, patents, and other references mentioned herein are incorporated by reference in their entirety. The particular embodiments discussed below are illustrative only and not intended to be limiting.

### BRIEF DESCRIPTION OF THE DRAWINGS

FIG. 1 is a graph showing quantification of the number of BrdU-positive neuroblasts in the subependymal zone (SEZ) of wild-type and lethally irradiated (LI) adult mice.

FIG. 2 is a graph showing the effect of LI on neurosphere (NS) cultures isolated from adult SEZ.

### DETAILED DESCRIPTION

The invention provides various methods for analyzing the effects of an agent on brain cells. One example of such a method involves comparing cells in the brain marrow of a test animal to which the agent has been administered to those in a control animal (matched in genetic background, age, sex, etc.) not administered the agent, but otherwise treated in an identical manner as the control animal. Another example of such a method involves comparing neural stem cell repopulation of a test animal to which an agent has been administered, to that in a control animal not administered the agent, but otherwise treated in an identical manner as the control animal.

The methods may be used to assess the potential of a wide variety of agents to impact the important ongoing process of neurogenesis in the brain, either positively or negatively. The methods can be used, for example, to analyze the ability of a test compound or treatment regimen to act as a neurotoxic agent, or to decrease neurogenesis. For example, the methods of the invention could be used to screen candidate therapeutic drugs (for example for disorders of nervous system such as Alzheimer's disease, Parkinson's disease, AIDS-related dementia and neurologic disorders, or other disorders) chemotherapeutic agents or protocols, food additives, nutritional supplements including herbal extracts, environmental agents, etc., for their potential to harm the cells of the brain marrow. The methods of the invention can also be used to identify new drugs, candidate compounds, natural extracts and the like, and treatments that can increase neurogenesis in an animal, and/or preserve or protect existing brain marrow stem and progenitor cells from a neurotoxic agent or protocol that can otherwise deplete the numbers of neurogenic cells or diminish the neurogenic capacity of the brain marrow.

The below described preferred embodiments illustrate adaptations of these methods. Nonetheless, from the description of these embodiments, other aspects of the invention can be made and/or practiced based on the description provided below.

### Assessing Agent-Induced Brain Marrow Changes

The invention provides a method of assessing the ability of an agent to affect brain cells and neuropoiesis by comparing the brain marrow of a test animal to which the agent has been administered to that in a control animal not administered the agent. In one embodiment of this method, an agent is administered to a test animal (for example, any suitable animal such as a rat or a mouse). The agent can be any substance or force that might have an effect on brain cells. For example, the agent may be a substance such as a small organic or inorganic molecule, a nucleic acid or a nucleoside analog, or a polypeptide; or the agent may be a force such as radiation (for example, ionizing radiation) or magnetic force.

One class of agents of interest are nucleoside analogs that act as DNA chain-terminating agents for example by inhibiting nucleoside or nucleotide reverse transcriptase enzymes. Such agents have found use as antiretroviral therapeutics for diseases such as HIV, and in some cases as chemotherapeutic agents. Agents of this class include but are not limited to drugs such as AZT (3-azido-3′-deoxythymidine), dideoxythymidine, dideoxycytidine, dideoxyinosine, cytosine arabinose, and lamivuidne (3TC), and variants thereof having DNA-chain terminating actions.

The agent can be administered to the animal by any suitable technique, for example, oral or parenteral administration such as by intravenous, intramuscular, or subcutaneous injection, or intracranial administration. Intracranial administration can be via any suitable route, for example by retro-orbital sinus injection, or by direct application to an appropriate region of the brain. Methods for delivery of an agent to a discrete area of the brain are well known in the art, and can include the use of stereotactic imaging and delivery devices.

The agent may be administered intermittently or continuously, and the route of administration may vary depending upon the purpose of the administration. A useful system for continuous administration of an agent is an osmotic minipump which may be implanted in the body of a test animal, for example subcutaneously. Use of osmotic minipumps for delivery of nucleoside analogs is further described in an example below.

At various times subsequent to administering the agent (for example, 1 h, 2 h, 6 h, 12 h, 1 d, 2 d, 3 d, 4 d, 5 d, 6 d, 7 d, 14 d, 21 d, 28 d or more), the cells in the SEZ, RMS, olfactory bulb and/or hippocampus are examined for changes relative to the same cells in the control animal (for example, an animal identical to the test animal except that it is not administered the agent). Examination of cells can be accomplished by in vivo imaging of the region, or by analyzing a brain tissue sample taken from the SEZ, RMS, olfactory bulb and/or hippocampus (the brain marrow). Suitable methods for isolating a brain tissue sample taken from the SEZ, RMS, olfactory lobe and/or hippocampus are known in the art. See, for example, Zheng et al., Cloning and Stem Cells 4:3-8, 2002; and Monje et al., Nature Medicine 8:955-962, 2002.

To identify an agent-induced change in the brain marrow, at least one characteristic of cells of the brain tissue sample is analyzed. A number of different characteristics of such cells might be assessed. For example, a phenotypic characteristic might be analyzed. Phenotypic characteristics include the mitotic index of cells in the sample (for example, as determined by bromodeoxyuridine (BrdU) labeling (see, for example, Larison and Bremiller, Development 109:567-576,1990; Hu and Easter, Dev. Biol. 207:309-321, 1999; and Gotz and Bolz, J. Neurobiol. 23:783-802, 1992) and marker expression (for example, of one or more of the following: microtubule associated protein (MAP), βIII tubulin, nestin, PSA-NCAM, NeuN, doublecortin, GFAP, 04, CNPase, and galactocerebroside).

Phenotypic characteristics of the brain tissue can also be assessed by electron microscopy (ultrastructurally), for example, to reveal evidence of necrosis and/or apoptosis in particular cell types of the brain marrow, such as the so-called type A, B and C cells of the SEZ.

As another example, a functional characteristic (i.e., the ability of the cells to do something) might be analyzed. For instance, the ability of the cells to form neurospheres in in vitro culture could be analyzed. See, for example, Laywell et al., Methods in Molecular Biology 198:15-27, 2002. The number of neurospheres formed, as well as the cellular makeup of each neurosphere might be analyzed. The culture can be examined for stem cells, neuronal cells and glial progenitor cells, and differentiated neurons and glia (see for example, Laywell et al., Methods In Molec. Biology 198:15-27, 2002).

The final step of this method involves comparing the characteristic of the cells from the test animal to those in a control animal. A difference in the characteristic between the test and control animals indicates that the agent affects brain cells. For example, if an agent causes a lower mitotic index in the test animal compared the control animal, or if the cells from the test animal form fewer neurospheres when placed under appropriate culture conditions than do cells from the control animal, then the agent negatively affects brain cells and neuropoiesis.

In a variation of the above method, effects of agents on neurogenesis can be analyzed by introducing stem cells into test animals. This method includes the steps of: (a) administering at least one stem cell to a test animal; (b) administering the agent to the stem cell or the test animal; (c) analyzing population of the test animal's brain marrow by the administered stem cells; and (d) comparing said population by stem cells in the brain marrow of the test animal to that in a control animal receiving at least one control stem cell, wherein neither the control stem cell nor the control animal were exposed to the agent.

A stem cell from any source capable of populating the brain marrow and behaving as a neural stem cell can be used. The stem cells can be neural stem cells, embryonic stem cells, or somatic stem cells derived from a non-neural tissue. Methods are described below for isolation of stem cells, including neural stem cells.

A sufficient dose of stem cells is in the range of one cell to hundreds of thousands of cells. Either before or after the step of administering the stem cells, the agent being tested is also administered to the animal or to the stem cells. If the stem cells are treated with an agent, this may be done, for example, by exposing the stem cells to the agent in tissue culture for various intervals (for example 1 h, 2 h, 6 h, 12 h, 1 d, 2 d, 3 d, 4 d, 5 d, 6 d, 7 d, 14 d, 21 d, 28 d or more) prior to implanting the stem cells in the test animal. If the animal is to be treated with the agent, the agent may be administered for various intervals to the test animal in vivo, for example by infusion using an osmotic minipump. At various times subsequent to administering the agent (for example, 1 h, 2 h, 6 h, 12 h, 1 d, 2 d, 3 d, 4 d, 5 d, 6 d, 7 d, 14 d, 21 d, 28 d or more), the stem cells are implanted, and the SEZ, RMS, olfactory bulb and/or hippocampus regions of the animal's brain are subsequently examined for population by the stem cells compared to that in a control animal receiving control stem cells (for example, an animal identical to the test animal except that it is not administered the agent, or the stem cells are not contacted with the agent).

Population of an animal's brain marrow by the introduced stem cells can be assessed in a variety of ways. For example, if the administered stem cells bear a detectable label, a brain tissue sample from the animal can be analyzed for the presence or quantity of the label as an indication that the stem cells have populated the brain marrow. As described in an example below, the administered stem cells can be genetically modified, for instance to express a marker such as green fluorescent protein (GFP) that renders the cells detectable, for instance, by fluorescence microscopy of brain sections following introduction of the cells into the brain (for example by transplantation). Additionally, the brain tissue sample might also be analyzed for morphological characteristics consistent with population by neurogenic cells, such as the presence of chains of proliferating cells (for example, as seen using BrdU labeling) or migrating cells in the RMS en route to the olfactory bulb, identified, for example, using a marker such as an antibody directed against polysialic acid neural cell adhesion molecule (PSA-NCAM) that selectively labels neuroblasts. A difference in the population of the brain by neurogenic cells and their progeny between the test animal and the control animal indicates that the agent affects brain cells and neuropoiesis.

### Assessing Neural Stem Cell Repopulation

Several variations of the methods of the invention include the step of depleting brain marrow in an animal, either to assess the ability of an agent to have a modulating affect on neurogenesis, or to increase neurogenesis following transplantation of stem cells. “Brain marrow repopulation,” as used herein, refers to the process of repopulating the neurogeneic stem cell population of the brain following depletion or elimination of endogenous neural stem and progenitor cells. By “depletion of brain marrow,” is meant reduction in the number of neural stem cells (neuroblasts) and their progeny in the brain marrow. Brain marrow can be depleted, for example, by irradiation. Methods for depletion of brain marrow using irradiation are known in the art. See, for example, Tada E et al., Exp. Neurol. 160:66-77, 1999; Monje M L et al., Nature Med. 8:955-962, 2002. Brain marrow can also be depleted using chemicals that block cell genesis, arrest mitosis, or specifically kill dividing cells in the brain marrow. Exemplary chemical compounds of this type include but are not limited to cytosine arabinoside, nucleoside derivatives, and the like.

An attempt is made to repopulate the brain marrow by administering a dose of stem cells normally sufficient to achieve the repopulation. A sufficient dose of neural stem cells is in the range of one cell to hundreds of thousands of cells. Optionally, as above, either before or after the step of administering the stem cells, the agent being tested is also administered to the animal or to the stem cells. At various times subsequent to administering the agent (for example, 1 h, 2 h, 6 h, 12 h, 1 d, 2 d, 3 d, 4 d, 5 d, 6 d, 7 d, 14 d, 21 d, 28 d or more), stem cells are introduced into animal and the SEZ, RMS, olfactory bulb and/or hippocampus regions of the animal's brain are examined for repopulation by the stem cells compared to that in a control animal (for example, an animal identical to the test animal except that it is not administered the agent, or the stem cells are not contacted with the agent).

Repopulation of an animal's brain marrow can be assessed in a variety of ways, in general as discussed above for assessing population of brain marrow following introduction of stem cells. For example, if the administered stem cells are genetically modified to express a detectable label such as GFP, a brain tissue sample from the animal can be analyzed for the presence or quantity of the label as an indication that the stem cells have repopulated the brain marrow.

A preferred embodiment of the method of increasing neurogenesis in an animal includes the steps of (a) depleting brain marrow in a test animal; (b) administering at least one stem cell to the test animal to repopulate the brain marrow of the animal; and (c) determining an increase in neurogenesis in the repopulated brain marrow compared with a control animal receiving stem cell therapy without depletion of its brain marrow.

The foregoing method can be used to identify agents that protect the brain marrow from becoming depleted in response to an insult. A candidate protective substance is administered to an animal either before or after a known brain marrow depleting agent is administered to the test animal. The candidate agent can also be administered to a stem cell that is subsequently administered to the animal. The results are compared to a control animal administered the known brain marrow depleting agent but not the candidate substance. Those candidate substances which prevent or reduce the amount of brain marrow depletion in the test animal compared to the control animal are those that exert a protective effect.

### Techniques for Isolating Stem Cells

Some variations of the methods of the invention involve isolating stem cells, including neural stem cells. Techniques for isolating stem/progenitor cells from a tissue sample such as a brain tissue sample include any number of well-known immunosorting or immunoseparating methods including fluorescence activated cell sorting (FACS). FACS involves labeling of cells with fluorochrome-conjugated antibody. The labeled cells are then analyzed and sorted on the basis fluorescent antibody staining using a flow cytometer. For use in FACS, any antibody that binds to a stem/progenitor cell-specific marker, e.g., CD15 (also known as 3-fucosyl-N-acetyl lactosamine and LeX/ssea-1 antigen), CD133 (AC 133), and lectin-bound glycoconjugates may be used (see, for example, Capela and Temple, Neuron 35:865-875, 2002; and Thomas L B et al., Glia 17:1-14, 1996).

Additionally, promoter driven enriching protocols (see for example, Roy et al., J. Neurosci. Res. 59:321, 2000; and Wang et al., Dev. Neurosci. 22:167, 2000) such as those using phentoypic promoters of neurons and glial cytoskeletal proteins such as nestin, GFAP, neuronal βIII tubulin and others that drive marker proteins such as GFP may be useful when performing a FACS analysis. See, for example, Capela and Temple, Neuron 35:865-875, 2002. For a review of FACS methods, see for example Herzenberg et al., Clin. Chem. 48:1819-1827, 2002. Techniques for sorting neural stem cells using FACS are described, for example in Murayama et al., J. Neurosci. Res. 69:837-847, 2002; and Ostenfeld et al., Brain Res. Dev. Brain Res. 134:43-55, 2002.

Other methods of separation can also be used such as high gradient magnetic cell sorting (magnetic activated cell sorting, MACS), immunopanning or selection after transfection with a promoter that drives a marker gene. Immunomagnetic separation and sorting techniques generally involve incubating cells with a primary antibody specific to a surface antigen found on the target cell type, immunologically coupling the target cells to magnetic beads (for example, anti-CD15 antibody conjugated to magnetic particles), and then separating the target cells out from the heterogeneous cell population using a magnetic field. A protocol for isolating progenitor cells using MACS is described, for example, in Martin-Henao et al., Bone Marrow Transplant 18:603-609, 1996. Immunomagnetic protocols are also described, for example, in Wright et al., J. Neurosci Methods 74:37-44,1997.

Immunopanning techniques involve the plating of a tissue culture dish with an antibody that binds a cell marker of interest, plating of cells onto the dish, washing away unbound cells, and isolating the antibody-bound target cells by trypsin digest. Immunopanning techniques are well known in the art and are described, for example, in Mi and Barres J. Neurosci. 19:1049-1061, 1999; Ben-Hur et al., The Journal of Neuroscience 18:5777-5788, 1998; Ingraham et al., Brain Res Dev Brain Res 112:79-87,1999; Murakami et al., J. Neurosci. Res. 55:382-393,1999; and Oreffo et al., J. Cell Physiol. 186:201-209, 2001.

Additionally, combinations of immunosorting and immunoseparating methods can be used to isolate stem/progenitor cells from a brain tissue sample. For example, magnetic microbead selection can be followed by an immunoadsorption technique, for example, using biotinylated antibody applied to a column of avidin-coated sephadex beads or an immunoaffinity column (Johnsen et al., Bone Marrow Transplant 24:1329-1336,1999; Lang et al., Bone Marrow Transplant 24:583-589, 1999; Handgretinger et al., Bone Marrow Transplant 21:987-993, 1998). Another example of a sorting technique involves use of a magnetic cell sorter followed by a selection step with an anti-stem/progenitor cell-specific marker antibody (e.g., anti-CD15) bound to immunomagnetic beads (Martin-Henao et al., Transfusion 42:912-920, 2002). A combination of two MACS systems may also be used in methods of the invention (Lang et al., Bone Marrow Transplant 24:583-589, 1999).

Conveniently, the antibodies used in the methods described above for isolating a stem/progenitor cell from a brain tissue sample are conjugated with labels to allow for ease of separation of the particular cell type, for example, using magnetic beads, biotin (which binds with high affinity to avidin or streptavidin), fluorochromes (which can be used with a FACS), haptens, and the like. Multi-color analyses may be employed with the FACS or in a combination of immunomagnetic separation and flow cytometry. Multi-color analysis is of interest for the separation of cells based on multiple surface antigens, for example, CD15, CD44, CD133, and lectin-bound conjugates, as well as non-surface phenotypic markers including neuronal versus glial cytoskeletal proteins as described above (for example, nestin, GFAP, and neuronal beta III tubulin). Fluorochromes which find use in a multi-color analysis include phycobiliproteins, for example, phycoerythrin and allophycocyanins, fluorescein and Texas red. A negative designation indicates that the level of staining is at or below the brightness of an isotype-matched negative control. A dim designation indicates that the level of staining may be near the level of a negative stain, but may also be brighter than an isotype-matched control.

Subsequent to isolating stem/progenitor cells and using these cells to culture neurospheres, low-power inverted phase microscopy is a useful technique for analyzing (for example, counting) neurospheres generated from a brain sample. In one method of counting neurospheres using low-power inverted phase microscopy, dissociated cells are quantified and plated at a known density (for example, 10,000 cells per well) in each well of a multi-well (such as a 6-well) culture plate. Growth factors are added at suitable intervals (for example, every second day for two weeks). Neurospheres are collected from each well, gently pelleted, and resuspended in fresh medium. An aliquot of each sample (for example, 50 μl) is placed on a slide (such as a glass slide), and the number of neurospheres is determined, for example with low-power inverted phase microscopy.

### Differentiated Neural Cell Lineages

Once isolated, brain cells may be analyzed for a number of characteristics, including cellular differentiation. To confirm the differentiation of cells into neurons, astrocytes and oligodendrocytes, the cells can be subjected for example to immunocytochemistry or reverse-transcriptase polymerase chain reaction (RT-PCR) to determine the presence of cell-specific markers (for example, GFAP for astrocytes, βIII tubulin for neurons, and O4 for oligodendrocytes). Antibodies specific for various neuronal or glial proteins may be employed to identify phenotypic properties of the differentiated cells. Neurons may be identified for example using antibodies to neuron-specific enolase, neurofilament, tau, βIII tubulin, or other known neuronal markers. Astrocytes may be identified for instance using antibodies to GFAP or other known astrocytic markers. Oligodendrocytes may be identified using antibodies to galactocerebroside, O4, myelin basic protein or other known oligodendrocytic markers. Glial cells in general may be identified by staining with antibodies, such as the M2 antibody, or other known glial markers.

It is also possible to identify cell phenotypes by identifying compounds characteristically produced by those phenotypes. For example, it is possible to identify neurons by the production of neurotransmitters such as acetylcholine, dopamine, epinephrine, norepinephrine, and the like.

### Animals

Because subjects of many different species have brain marrow and neurological disorders, the invention is believed to be compatible with many types of animal subjects. A non-exhaustive exemplary list of such animals includes mammals such as mice, rats, rabbits, goats, sheep, pigs, horses; cattle, dogs, cats, and primates such as monkeys, apes, and human beings. In the experiments described herein, the subjects used were mice. Nonetheless, by adapting the methods taught herein to other methods known in medicine or veterinary science (for example, adjusting doses of administered substances according to the weight of the subject animal), the assays of the invention can be readily optimized for use in other animals.

### EXAMPLES

General Comment:

The following materials and methods were used in the practice of the invention described in Examples below.

Animals. Female C57BL/6 mice (over 3 months of age) were used as a model system, and were housed at the University of Florida's Department of Animal Care Services, in compliance with IACUC regulations.

Irradiation and bone marrow reconstitution. Animals were placed in individual chambers of a plexi-glass container for irradiation. Lethal irradiation (LI) was induced by exposure to a Cs¹³⁷ source in a Gamma Cell 40 irradiator until 850 Rads had been obtained. This amount of radiation is sufficient to deplete the bone marrow of viable cells, while not inducing immediate death. Immediately following irradiation, LI mice were anesthetized with isofluorane and a “rescue dose” of 1×10⁶ whole bone marrow (WBM) cells was administered to each mouse via retro-orbital sinus (ROS) injection to promote survival and repopulation of the erythropoietic system. WBM was isolated from the femurs of a sacrificed litter mate (anesthetized by exposure to isofluorane then sacrificed by cervical dislocation), washed in 10 ml of phosphate buffered saline (PBS), and re-suspended in an appropriate volume of PBS after quantifying with a hemacytometer. A 32 gauge needle attached to a 1 ml insulin syringe was inserted into the ROS and the WBM was injected in a volume of 150 μl. Animals were allowed to recover before being returned to conventional animal housing.

Tissue immunohistochemistry. Both wild type (WT) and LI animals were given a lethal dose of the anesthetic Avertin, prepared using tertiary pentyl alcohol and tribromoethanol before being perfused through the left ventricle with 4% paraformaldehyde (PFA) in PBS. Following perfusion, the brain was removed, post-fixed overnight in 4% PFA at 4° C., then serially sectioned through either the coronal or sagittal plane at 40 μm using a Leica vibratome (model VT-1000-S) equipped with a sapphire blade. Tissue was prepared for immunohistochemistry by blocking at room temperature for 1 hour in PBS containing 10% fetal bovine serum, 5% dry milk, and 0.01% Triton X-100. Primary antibodies (Monoclonal Anti-β-III Tubulin: Promega, Madison, Wis.; G712A, 1:1000; Polyclonal Anti-β-III Tubulin: Covance, Princeton, N.J.; PRB435P, 1:5000; Monoclonal Anti-Polysialic Acid Neural Cell Adhesion Molecule [PSA-NCAM]: Chemicon, Temecula, Calif.; MAB5324, 1:100; Monoclonal Anti-BrdU: Human Hybridoma Bank, Manassas, Va.; 1:30; Polyclonal Anti-Glial Fibrillary Acidic Protein [GFAP]:Immunon Shandon, Pittsburgh, Pa.; 1:100) were applied to the sections overnight with moderate agitation at 4° C.

Residual primary antibody was removed by three 5 minute washes (PBS plus 0.01% Triton X-100), and secondary antibodies (Rhodamine Red-X, Goat Anti-Mouse IgG: Molecular Probes, Eugene, Oreg.; R-6393, 1:500; Texas Red Anti-Rabbit IgG, Vector Labs; TI-1000, 1:500; Fluoroscein Anti-Rabbit IgG, Vecor Labs, Burlingame, Calif.; FI-1000, 1:500) were applied at room temperature for 50 minutes. Finally, sections were washed in PBS three times for 5 minute, mounted on positively charged glass slides (Fisherbrand, Pittsburgh, Pa. Superfrost/Plus™, 12-550-15), and allowed to dry for 15 minutes at 37° C., before being cover-slipped in Vectashield (Vector Labs, Burlingame, Calif.; H-1000) mounting medium. Sections were analyzed and photographed by fluorescence microscopy using either a Zeiss (Thornwood, N.Y.) Axioplan 2 upright microscope or a Leica (Wetzlar, Germany) Model DMLB.

Identification of proliferative cells by BrdU labeling. Three weeks or 3 months following lethal irradiation, WT and LI mice received 5-bromo-2′-deoxyuridine (BrdU, Sigma, St. Louis, Mo.; B-5002) three times a day for three days via intra-peritoneal (IP) injection (3 μg/300 μl per injection). The brains were fixed, removed, and sectioned as above. Sections were prepared for BrdU immunohistochemistry by first incubating in 2×SSC/Formamide solution (1:1) for 2 hours at 65° C. After washing in 2×SSC for 5 minutes at room temperature, sections were then incubated in 2N HCl for 30 minutes at 37° C. Finally, sections were washed in 0.1M borate buffer for 10 minutes at room temperature, and then processed for double immunolabeling with monoclonal anti-BrdU antibody and polyclonal anti-β-III tubulin, as described above.

Serial coronal sections were analyzed for BrdU positive cells in the SEZ using a blind study format (sections coded and scored by separate investigators). The region of the SEZ analyzed encompassed an area extending from the inferior tip of the lateral ventricle, superiorly along the wall of the lateral ventricle (an area approximately 5 cell bodies deep) to a point extending 700 μm from the dorsolateral corner of the lateral ventricle. More particularly, sections were analyzed at 40× magnification for the presence of BrdU positive neuroblasts. The region of analysis was conserved between all sections (3 adjacent sections per animal). Candidate adjacent sections were those where the anterior commissure (AC) was just lateral of the ventral most point of the lateral ventricle (LV). The area analyzed was composed of a region that began at the ventral-most portion of the LV, extended along the wall of the lateral ventricle, then proceeded 700 um laterally from the lateral horn. The region along the wall of the LV extended 5 cells deep into the striatum (ST). Cells in both focal planes of the tissue were analyzed, and the total number was calculated. BrdU positive (red stained) cells lining the wall of the lateral ventricle also stained positive for β-III tubulin (green). The defined region was carefully analyzed at a magnification of 40×, and at both focal planes of the tissue.

To maintain consistency between sections, BrdU labeled cells were scored as “positive,” regardless of the intensity of the antibody fluorescence. Three adjacent sections per tissue were scored, using the location of the anterior commissure as a consistent landmark between sections. The cell numbers were collected, averaged, and placed into a graphical format using a Microsoft (Redmond, Wash.) Excel™ spreadsheet. Statistical significance of values was determined by paired student-t test analysis, with P-values less than 0.05 deemed to be significant.

Cell culture. For comparative analysis of the effect of lethal irradiation on the neural stem cell (NSC) population of the SEZ, neurosphere (NS) cultures were generated from WT and Li animals, as described (Laywell E D et al., In: Zigova T et al. eds. Methods in Molecular Biology, Vol. 198, Neural Stem Cells; Methods and Protocols, Humana Press Inc., Totowa N.J. 2002, pp. 15-27.) Briefly, animals were anesthetized with isofluorane and decapitated. The brain was removed and placed on an ice-cold sterile dissection board, and a rectangular forebrain block containing the SEZ was obtained by removing the olfactory bulb, cerebellum, hippocampus, lateral portions of the striatum and lateral and dorsal cerebral cortex. The block was minced with a sterile scalpel, and placed in ice-cold PBS containing antibiotic and anti-mycotic agents (Penicillin-Streptomycin, Gibco/Invitrogen, Carlsbad, Calif.; 15140-122, and Fungizone Antimycotic, Gibco Invitrogen, Carlsbad, Calif.; 15295-017) for 10 minutes.

Minced tissue was then centrifuged for 5 minutes at 1100 rpm at 4° C., re-suspended in 3 mL 0.25% Trypsin plus EDTA (Gibco/Invitrogen, Carlsbad, Calif.; 25200-056), then incubated at 37° C. for 5 minutes. After neutralizing the trypsin by the addition of 1 mL of fetal bovine serum, the tissue was triturated into a single cell suspension by pipetting through a series of descending diameter, fire-polished Pasteur pipettes. The cells were washed in DMEM/F-12 (Gibco/Invitrogen, Carlsbad, Calif.; 11330-032) at 1100 rpm for 5 minutes at 4° C., and re-suspended in growth medium consisting of DMEM/F-12, 5% FBS, L-Glutamine (Gibco/Invitrogen, Carlsbad, Calif.; 25030-081), N-2 supplement, (Gibco/Invitrogen, Carlsbad, Calif.; 17502-048), recombinant-human-EGF (20 ng/mL, R&D Systems, Minneapolis, Minn.; 236-EG), and recombinant-human-FGF (10 ng/mL, R&D Systems, Minneapolis, Minn.; 233-FB). The cells were plated out in non-adhesive 6-well plates (Costar, Kennebunk, Me.; 3471) at a density of 1000 cells/cm². Cultures were supplemented with EGF and FGF (20 ng/mL and 10 ng/mL, respectively), every second day.

Double-blind analysis of the effects of LI on neurosphere yield. In order to determine the effects of lethal irradiation on NS generation, we used a double-blind paradigm to enable unbiased preparation and examination of the cultures from three WT and three LI mice (two months following lethal irradiation, age matched). Briefly, the animals were sacrificed and their brains removed by investigator A, who gave each brain an identifying number (1 through 6). The brains were then given to investigator B who removed the SEZ (as described above) from each brain in an identical fashion. The isolated SEZ tissue was then re-coded with a letter (A through F) by investigator C, who remained the only individual to know both the letter and number code. The tissue was then returned to investigator A for culture (as described above) and quantification.

At 14 days in vitro, NS were collected, pelleted, and re-suspended in 2 mL of media. To determine NS yield, four 50 μl aliquots from each culture were placed in a 12-well tissue culture plate. The aliquots were analyzed with a Nikon inverted phase microscope at both 4× and 10× magnifications, with NS diameter determined by use of the “SPOT™” program. NS below 40 nμm in diameter were excluded as a means to avoid potential confusion with hypertrophied cells. Additionally, spherical aggregates that did not display the NS criteria of a tight phase contrast-bright perimeter were not counted. The total number of spheres per aliquot was determined, and the total yield and percent yield of each culture was then calculated from these numbers. Statistical significance of values was determined by paired student-t test analysis, with p-values less than 0.05 deemed to be significant. At the conclusion of the analysis, the code was broken and the identity of the cultures was revealed.

Immunohistochemical analysis of neurospheres cultured from wild type and LI mice. NS were picked from their cultures using a handheld pipetter set at 2 μl and placed in DMEM/F-12 plus 5% FBS atop a laminin/poly-D-lysine coated, chambered culture slide (Becton/Dickinson, Palo Alto, Calif.; Catalog number 352688). Spheres were allowed to attach and differentiate for 2 days, at which time the media was removed and the cells were fixed by incubation in 4% PFA in PBS at room temperature for 30 minutes. After fixing, the cells were processed for immunolabeling with antibodies against β-III tubulin and GFAP, as above.

### Example 1

### Isolating Brain Marrow from an Animal

A portion of an animal brain (e.g., RMS, SEZ, hippocampus) can be surgically removed and mechanically separated into smaller tissue pieces. One such method for mechanically separating a brain sample into smaller tissue pieces involves mincing the brain sample with a razor blade. To further dissociate the pieces into a single cell suspension, the tissue pieces are incubated in a solution containing a proteolytic enzyme such as trypsin, papain and/or hyaluronidase. An example of such an incubation involves incubating tissue pieces in a trypsin/EDTA solution for 10 minutes at 37° C. Other examples include an incubation in 14 U/ml of papain or 1.3 mg/ml trypsin and 0.67 mg/ml hyaluronidase for 1 hour with gentle rocking.

Following incubation with an enzyme-containing solution, the tissue may be subjected to further mechanical dissociation using a Pasteur pipette (e.g., fire-polished Pasteur pipette). Once the tissue is dissociated into a single cell suspension, the cells are washed with a suitable medium (e.g., washed twice with basal medium containing N2 supplement (Invitrogen, Carlsbad, Calif.; Cat.#17502048,) and 5-10% fetal bovine serum (FBS), or washed with DMEM (Gibco, Carlsbad, Calif.)). After washing, the tissue is then resuspended in a suitable medium (e.g., N2-supplemented medium containing 5% FBS, 20 ng/ml EGF and 20 ng/ml bFGF or DMEM), resulting in a single cell suspension. Cells in the brain tissue sample are then cultured under conditions appropriate for culturing neurospheres as described below.

### Example 2

### Neurosphere Formation

Typically, isolated neural cells are cultured in a medium that permits the growth and proliferation of neurospheres. The culture in which the isolated cells proliferate can be a serum-free medium containing one or more predetermined growth factors effective for inducing multipotent neural stem cell proliferation. The culture medium can be supplemented with a growth factor selected from leukocyte inhibitory factor (LIF), epidermal growth factor (EGF), basic fibroblast growth factor (FGF-2; bFGF) or combinations thereof. The culture medium can be further supplemented with neural survival factor (NSF) (San Diego, Calif.) and/or fetal bovine serum. Neurospheres cultured according to this method are not immunoreactive for glial fibrillary acidic protein (GFAP; a marker for astrocytes), neurofilament (NF; a marker for neurons), neuron-specific enolase (NSE; a marker for neurons) or myelin basic protein (MBP; a marker for oligodendrocytes). However, cells within the neurosphere are immunoreactive for nestin, an intermediate filament protein found in many types of undifferentiated CNS cells (Lehndahl et al., Cell 60: 585-595, 1990).

The identification, culture, growth, and use of mammalian, including human, neural stem cell cultures, either as suspension cultures or as adherent cultures, is disclosed in Weiss et al., U.S. Pat. No. 5,750,376 and Weiss et al., U.S. Pat. No. 5,851,832, Published International Applications WO/98/30678 and WO/02/14479 and Laywell et al., Methods in Molecular Biology 198:15-27, 2002. Kukekov et al., U.S. Pat. No. 6,638,763 discloses a suspension culture method using methylcellulose or other factors that deter cell-cell, and cell-substrate interactions. Additionally, Johe, U.S. Pat. No. 5,753,506 refers to adherent CNS neural stem cell cultures.

### Example 3

### Analyzing Effects Of Agents on Brain Cells

Certain brain marrow constituents have been previously characterized. It has been established that the periventricular subependymal zone (SEZ) of the adult mouse brain exhibits persistent expression of developmentally regulated molecules, including the extracellular matrix (ECM) protein tenascin that delineates the rostral migratory stream (RMS), extending from the lateral ventricle to the olfactory bulb (OB). This dense ECM within the brain marrow, similar to what is observed in bone marrow, can be visualized in brain sections, for example using an antibody to tenascin. Marrow tissues are characterized as exhibiting persistent cell proliferation, which in the case of brain marrow reflects ongoing neurogenesis.

Immunostaining of sagittal brain sections including the SEZ from control adult mice using an antibody against neuronal β-III tubulin revealed densely packed immunopositive immature neurons having few processes, oriented postero-laterally as they migrated through the brain marrow to the olfactory bulb. In mice injected systemically with the cell proliferation marker brdU 3 hours prior to sacrifice, cells immunopositive for neuronal β-III tubulin were also positive for BrdU, demonstrating proliferation of the immature neurons.

Brain marrow was observed in a similar manner in age-matched adult mouse 7 days after irradiation, performed as described above. In parasagittal sections through the forebrain, immunostaining with β-III tubulin antibody was absent in the SEZ/RMS following irradiation. Thus neurogenesis was greatly diminished in these animals by the radiation treatment.

### Example 4

### Examining Repopulation Of Stem/Progenitor Cells

A mouse received a brain marrow-depleting sublethal irradiation protocol as described above. Green fluorescent protein-labeled (GFP) neural stem cells were transplanted into the ventricular system of the mouse. High and low magnification images of the transplanted neural stem cells were examined.

The results showed that GFP-labeled cells insinuated themselves back into the SEZ, and began to migrate as neuroblasts in the RMS. Additionally, GFP-expressing stem cells were observed that homed to the irradiation-depleted hippocampus and began to functionally integrate into that location. One such cell took up a normal position and differentiation program within the hippocampal dentate gyrus of an irradiated animal. Thus it was possible to repopulate the depleted brain marrow by transplantation of neural stem cells.

### Example 5

### Depletion of Neurogenesis in SEZ by Whole Body Irradiation

This example shows the effects on migrating neuroblasts of the mouse brain of an agent (i.e, a single lethal dose of ionizing radiation) on the SEZ, RMS and OB.

Mice were subjected to lethal doses of x-irradiation, then supplied with a rescue dose of wild-type bone marrow to allow for recovery of the ablated hematopoietic system. At two weeks post-LI, the animals were perfused with 4% paraformaldehyde and their brains sectioned into 40 μm thick sagittal sections. Antibodies against the migrating neuroblast specific marker PSA-NCAM were applied to the tissue, and the sections were photographed at 20× magnification.

In wild-type (WT) control animals, PSA-NCAM positive neuroblasts were abundant, and could be seen extending from the ventricle to the olfactory bulb. In these sections, the migration of neuroblasts through the RMS from the subependymal zone to the olfactory bulb was visible as a robust chain of PSA-NCAM positive cells.

Two weeks following lethal irradiation (LI), irradiated mice showed a marked decrease in the number of PSA-NCAM positive neuroblasts in the RMS. Photographs of sections of these brains revealed depletion of migrating neuroblasts from the RMS of LI mice. This observation was corroborated following staining of tissue sections from the brains of both WT and LI mice with the pan-neuronal marker β-III tubulin. Neuroblast depletion was variable, with some animals retaining small pockets of cells in the RMS. However, the overall abundance of migrating neuroblasts in the RMS of LI mice was never similar to those seen in untreated mice. Furthermore, the volume of migrating neuroblasts did not recover to the level seen in control animals, even at three months post-LI. These findings led us to the conclusion that exposure to ionizing radiation results in permanent damage to the neuroblast-producing cells of the SEZ.

Analysis and quantification of neuroblast depletion in the SEZ. To quantify the degree of neuroblast depletion induced by LI, we used 5-bromo-2′-deoxyuridine (BrdU) to label mitotic cells within the SEZ (Thomas M A et al., Glia 17:1-14, 1996). Briefly, 3 weeks and 3 months following LI (n=4 for each condition), treated and un-treated mice were given BrdU intra-peritoneally three times a day for three days, then sacrificed. The SEZ in WT animals, when immunolabeled with antibodies against β-III tubulin and BrdU on serial coronal sections at the level of the anterior commisure (AC), contained a robust layer of dividing neuroblasts. Three weeks following LI, this same region exhibited a significant decrease in newly-generated neuroblasts, and this depletion persisted at 3 months post LI.

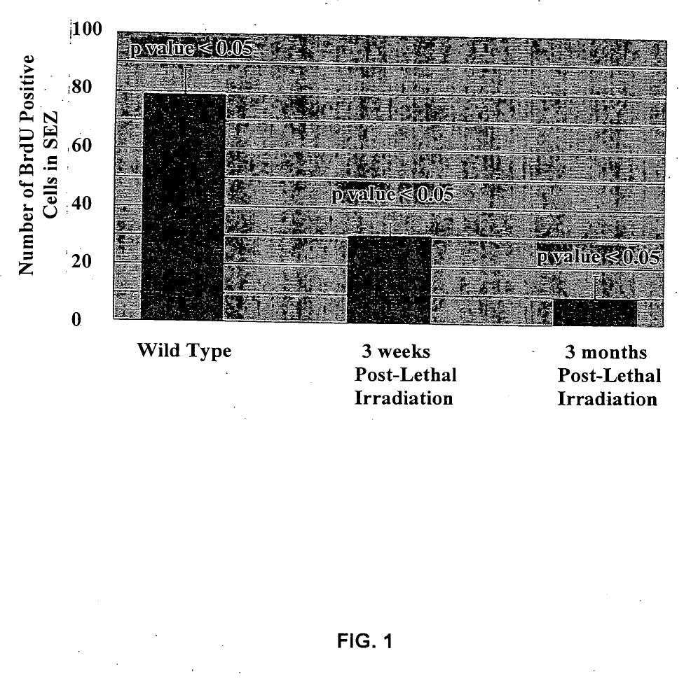

FIG. 1 is a graphical representation of the number of BrdU-positive cells for each condition (4 mice per condition, 3 sections per animal). The total number of BrdU positive neuroblasts in three adjacent coronal sections of either wild-type or LI mice was tabulated and placed into the above graphical format.

We observed a decrease of approximately 60% in the number of BrdU-positive cells at the 3-week time point as compared to the control. Paired student-t test analysis confirmed that this decrease is significant (p value=0.003). There was an 87% decrease in the number of BrdU-positive cells at the 3-month time point as compared to control, a significant value as determined by student-t test (p value=0.002). The 68% decrease in the number of BrdU-positive cells between 3 weeks and 3 months post LI was also significant (p value=0.03).

β-III tubulin immunolabeling confirmed that the cells scored were, in fact, neuroblasts rather than other mitotic cells residing in this area. BrdU-positive cells stained positive for β-III tubulin in both the control and 3 month post-LI mice. This data confirms that the decrease in migrating neuroblasts in the RMS is reflected in the SEZ, and that this depletion is significant and long-term.

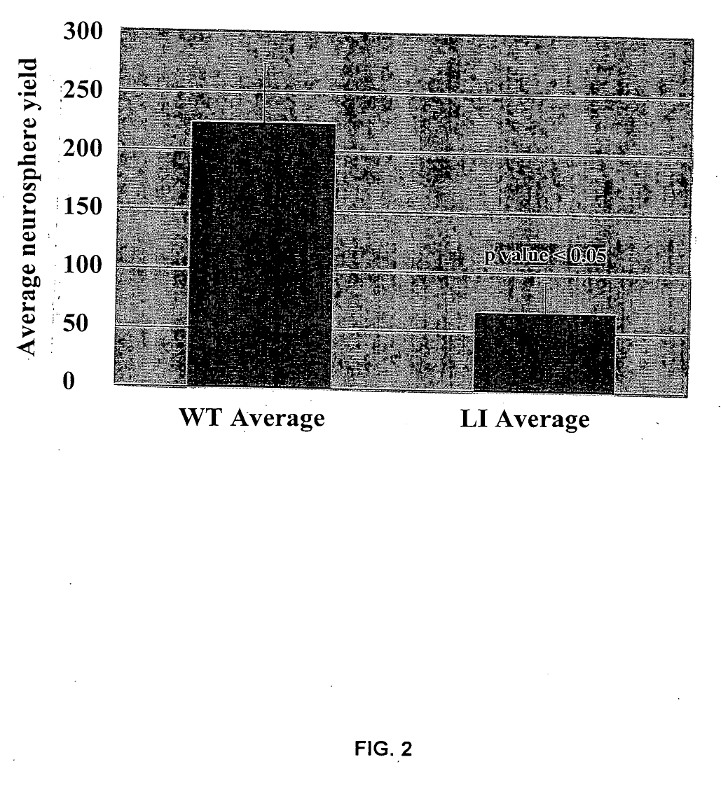

Effects of LI on neurosphere yield in culture. As it is well accepted that the neurosphere-forming cell is the in vitro manifestation of the neural stem cell (Reynolds B A and Weiss S, Science 255:1701-10,1992), we cultured neurospheres from both WT and LI mice in a double-blind-study format to determine if the stem cell pool in the SEZ was affected by the LI. SEZ tissue was cultured to generate NS from both wild-type adult mice and adult mice LI two months prior, with all tissues treated in identical fashion. The resulting NS yield was determined according to the protocol described above. Referring to FIG. 2, the results showed that neurospheres cultured from LI brains displayed an average decrease in yield of approximately 77%, when compared to the WT cultures (p value of 0.02). This decrease closely corresponds to the decreased levels of BrdU-positive neuroblasts in vivo following lethal irradiation, further supporting the validity of this finding.

It has been reported that the stem cell population of the SEZ is between approximately 0.02% and 1.0% of the total cells (Reynolds B A and Weiss S, Science 255:1701-10,1992; Reynolds B A and Weiss S, Dev. Biol. 175:1-13,1996; Weiss S. et al., J. Neurosci. 16:7599-7606, 1996), as determined by neurosphere yield from dissociated SEZ tissue, and the average yield of neurosphere from the WT brains in the present study falls within this range (0.15%). The average yield of NS isolated from the lethally irradiated brains was significantly lower, at 0.03%, indicating that exposure to lethal doses of radiation depletes the number of NSC in the brain responsible for the generation of NS.

### Example 6

### Effects of Antiretroviral Nucleoside Analogs on Neurogenesis

The commonly prescribed antiretroviral drug AZT (3′-azido-3′-deoxythymidine), as well as other similar DNA chain terminating agents such as dideoxythymidine, dideoxycytidine, dideoxyinosine, and 3TC may have deleterious effects on persistent neurogenesis within the hippocampus and subependymal zone (SEZ) of adult mammalian brains. AZT and the other above-mentioned drugs are marketed as selective reverse transcriptase inhibitors, but all work by terminating chain formation when they are inserted by any DNA polymerase into elongating DNA chains.

The effect of DNA chain terminating agents (nucleoside analogs) such as AZT on neurogenesis is tested via the following experimental paradigm. Four groups of adult mice (n>4 for each group) are administered 0, 10, 50, or 100 mg/kg/day AZT via Alzet osmotic minipumps implanted subcutaneously (Cat. #2004, Durect Corp., Cupertino, Calif.). These pumps release 0.25 microliters of solution per hour for 28 days. AZT is dissolved to the appropriate concentration in 0.9% sterile saline. Control animals receiving 0 mg/kg/day AZT are implanted with minipumps filled with sterile saline only. Under Avertin anesthesia, filled pumps are implanted into a subcutaneous pocket under the skin of the animal's back, where they remain for the duration of the experiment.

After 28 days (the end of the pump's infusion capacity) the animals receive a single injection of bromodeoxyuridine (BrdU) to label proliferative cells, e.g., within the SEZ and hippocampus. After an additional week, animals are transcardially perfused with buffer containing 4% paraformaldehyde, and their brains are removed and sectioned through the parasagittal plane with a microtome. Selected sections through the dentate gyrus of the hippocampus (the ultimate destination of newly-generated hippocampal neurons) and SEZ are immunolabeled for BrdU and a neuron-specfic protein such as NeuN or βIII tubulin, and the number of newly-generated neurons (double labeled for both markers) is quantified as described above. Additionally, some SEZ samples are analyzed for ultrastructural integrity using electron microscopy (EM). The fine structure of the SEZ has been well-established, and EM analysis is useful for detecting damage to this neurogenic region. In particular, deleterious effects of AZT are apparent by necrosis or apoptosis of the SEZ “A” cells, which represent neuronal precursors born in the SEZ that ultimately migrate through the rostral migratory stream to the olfactory bulb. Furthermore, damage to or loss of other SEZ cellular components (i.e., the “B” and/or “C” cells) is apparent from accumulated cellular debris in the area immediately subjacent to the ependymal cell layer.

In addition to the quantification of newly-generated neurons in fixed tissue sections, the relative ability to generate neurospheres from the brains of animals in the four experimental groups is also assessed. As described above, neurospheres are considered to be the in vitro correlate of in vivo neural stem cells. Damage to the cells responsible for neurogenesis in the brain therefore is likely to be apparent as a reduced capacity for neurosphere generation in the culture dish. At the end of the 28 day drug delivery period, animals are lightly anesthetized with isofluorane, and decapitated. The SEZ and hippocampus from each animal is gross-dissected, and cultured under neurosphere-generating conditions (see Example 2 above). The neurosphere yield from each brain is determined (see Example 5 above) in order to reveal differences in neurosphere-generating capacity among the experimental conditions.

### Other Embodiments

It is to be understood that while the invention has been described in conjunction with the detailed description thereof, the foregoing description is intended to illustrate and not limit the scope of the invention, which is defined by the scope of the appended claims. Other aspect, advantages, and modifications are within the scope of the following claims.

1. A method for analyzing a modulating effect of an agent on brain cells, the method comprising the steps of:
(a) administering the agent to a test animal;
(b) determining at least one characteristic of cells of brain tissue of said animal in an area selected from the group consisting of: subependymal zone, continuous rostral migratory stream, olfactory bulb and hippocampus; and
(c) comparing the at least one characteristic of the cells to that characteristic in cells of brain tissue in the same area in a control animal that was not administered the agent, wherein a difference in the characteristic between the test animal and the control animal indicates that the agent has a modulating effect on brain cells.

2. The method of claim 1, wherein the agent comprises at least one of a drug, a cell, a small molecule, a peptide, a nucleic acid, or a nucleoside analog.

3. The method of claim 2, wherein the nucleoside analog is selected from the group consisting of azidothymidine, dideoxyinosine, dideoxythymidine, dideoxycytidine, and cytosine arabinoside.

4. The method of claim 1, wherein the agent comprises radiation.

5. The method of claim 1, wherein the agent modulates neurogenesis in said brain tissue, or in a cell derived from said brain tissue.

6. The method of claim 1, wherein the characteristic is selected from the group consisting of mitotic index, expression of cellular markers, migration of neuroblasts in the brain marrow, apoptosis and necrosis.

7. The method of claim 1, wherein a sample of said brain tissue is removed from said animal.

8. The method of claim 7, wherein said sample is dissociated and placed into tissue culture.

9. The method of claim 8, wherein the tissue culture is placed under conditions that promote neurosphere formation.

10. The method of claim 9, wherein the step (b) of determining at least one characteristic of cells comprises analyzing the numbers of neurospheres formed in the tissue culture.

11. The method of claim 9, wherein the step (b) of determining at least one characteristic of cells comprises analyzing the cellular makeup of neurospheres formed in the tissue culture.

12. The method of claim 9, wherein the step (b) of determining at least one characteristic of cells comprises detecting expression of a cell-specific marker for at least one cell type consisting of a neuron, an astrocyte or an oligodendrocyte.

13. A method for analyzing the effect of an agent on neurogenesis in an animal, comprising the steps of:
(a) administering at least one stem cell to a test animal;
(b) administering the agent to the stem cell or to the test animal;
(c) analyzing population of the test animal's brain marrow with the administered stem cells; and
(d) comparing said population in the test animal to that in a control animal administered with a control stem cell, neither of said control stem cell or said control animal having been administered the agent, wherein a difference in the population by stem cells between the test animal and the control animal indicates that the agent has an effect on neurogenesis in the animal.

14. The method of claim 13, wherein the stem cell comprises a detectable label.

15. The method of claim 14, wherein the step (c) or (d) of analyzing population of the test or control animal's brain marrow is performed by quantifying the amount of detectable label associated with at least one tissue of the test or control animal selected from the group consisting of the SEZ, continuous RMS, olfactory lobe and hippocampus.

16. The method of claim 13, wherein the stem cell is selected from the group consisting of a neural stem cell, a somatic stem cell from a non-neural tissue, and an embryonic stem cell.

17. The method of claim 13, wherein the stem cell expresses a cell surface marker selected from the group consisting of CD15, CD133 and CD44.

18. The method of claim 13, wherein the stem cell is genetically modified.

19. The method of claim 13, further comprising the steps of depleting the brain marrow in the test and control animals, and comparing repopulation of the test animal's and control animal's brain marrow by the administered stem cells.

20. A method for increasing neurogenesis in an animal, comprising the steps of:
(a) depleting brain marrow in a test animal;
(b) administering at least one stem cell to the test animal to repopulate the brain marrow of said animal; and
(c) comparing the repopulation of the brain marrow in the test animal to that in a control animal receiving at least one stem cell without depletion of its brain marrow, wherein the presence of a greater number of neurogenic cells in said test animal indicates that neurogenesis is increased.

21. The method of claim 20, wherein the stem cell expresses a cell surface marker selected from the group consisting of CD15, CD133 and CD44.

22. The method of claim 20, wherein the stem cell is genetically modified.

23. The method of claim 20, wherein the stem cell is selected from the group consisting of a neural stem cell, a somatic stem cell from a non-neural tissue, and an embryonic stem cell.

24. The method of claim 20, wherein brain marrow is depleted by radiation or chemical means.

25. The method of claim 20, wherein the stem cell comprises a detectable label.
